(19) United States
(12) Patent Application Publication
(10) Pub. No.: US 2026/0283547 A1
(43) Pub. Date: Sep. 24, 2026

(54) DATA PROCESSING SYSTEM FOR CONVERTING MERIDIAN PARAMETERS INTO BIOMEDICAL DATA

(71) Applicants: Hsiang-Wei HU, Tainan City (TW); Rong-Shean LEE, Tainan City (TW)

(72) Inventors: Hsiang-Wei HU, Tainan City (TW); Rong-Shean LEE, Tainan City (TW)

(21) Appl. No.: 19/687,481

(22) Filed: May 26, 2026

Related U.S. Application Data
(63) Continuation-in-part of application No. 18/630,295, filed on Apr. 9, 2024.

(30) Foreign Application Priority Data
Apr. 10, 2023 (TW) 112113333

(51) Int. Cl. A61B 5/00; A61B 5/024; G16H 20/90

(52) U.S. Cl. CPC A61B 5/4854; A61B 5/0022; A61B 5/02438; A61B 5/7267; A61B 5/7275; A61B 5/742; G16H 20/90

(57) ABSTRACT

A data processing system includes a plurality of sensors, a measurement unit, an operating unit and a display. The sensors are attached onto a body of a subject at a plurality of sensing zones to generate a plurality of sensing signals. The measurement unit in communication with the plurality of sensors measures the plurality of sensing signals and generates a parameter set containing a plurality of meridian signal values. The operating unit in communication with the measurement unit receives the parameter set and performs an operation of the parameter set to generate a set of biomedical data, wherein the operation includes mapping a meridian feature space to a Western medical system space with a mapping weight matrix. The display in communication with the operating unit shows the set of biomedical data.

### FIELD OF THE INVENTION

The present invention relates to a data processing system, and more particularly to a data processing system, which converts meridian parameters into biomedical data.

### BACKGROUND OF THE INVENTION

Please refer to FIG. 1, which schematically illustrates a flow of a conventional medical service. After checking in (Step 11), the patient proceeds to inquiry by medical personnel (Step 12) to provide basic state of illness. General physiological data of the patient, e.g., body temperature, heart rate and respiratory rate, are then collected with simple instruments (Step 13) and interpreted by medical personnel (Step 14). Afterwards, medical personnel may require further inspection with advanced apparatus, such as biochemical analyzer, ultrasonic image analyzer, etc., based on his or her subjective interpretation of the collected data (Step 15). Once possible disease is diagnosed according to the inspection result, proper treatment and corresponding health education may then follow (Step 16). It is understood that the advanced inspection option in Step 15 highly relies on personal experience and subjective judgement of medical personnel in Step 14. Therefore, not all the clinics are capable of completely executing such procedures.

### SUMMARY OF THE INVENTION

Therefore, the present invention provides a data processing system, which can readily provide objective data for medical personnel to take proper actions.

In an aspect of the present invention, a data processing system includes: a plurality of sensors to be attached onto a body of a subject at a plurality of sensing zones to generate a plurality of sensing signals; a measurement unit in communication with the plurality of sensors for measuring the plurality of sensing signals and generating a parameter set containing a plurality of meridian signal values; an operating unit in communication with the measurement unit, receiving the parameter set and performing an operation of the parameter set to generate a set of biomedical data, wherein the operation includes mapping a meridian feature space to a Western medical system space with a mapping weight matrix; and a display in communication with the operating unit for showing the set of biomedical data.

### BRIEF DESCRIPTION OF THE DRAWINGS

The above contents of the present invention will become more readily apparent to those ordinarily skilled in the art after reviewing the following detailed description and accompanying drawings, in which:

FIG. 1 is a scheme illustrating a flow of a conventional medical service;

FIG. 2A is a functional block diagram schematically illustrating a data processing system according to an embodiment of the present invention;

FIG. 2B is a functional block diagram schematically illustrating a data processing system according to another embodiment of the present invention;

FIG. 2C is a functional block diagram schematically illustrating a model developed for the artificial intelligence (AI) interpretation engine according to an embodiment of the present invention;

FIG. 2D is a functional block diagram schematically illustrating a multi-task output unit according to an embodiment of the present invention;

FIG. 3 is a scheme illustrating a flow of a medical service according to an embodiment of the present invention;

FIG. 4 is a schematic diagram illustrating an example of the human-computer user interface of the data processing system according to the present invention;

FIG. 5 is a schematic diagram illustrating another example of the human-computer user interface of the data processing system according to the present invention;

FIG. 6 is a schematic diagram illustrating a further example of the human-computer user interface of the data processing system according to the present invention;

FIG. 7 is a schematic diagram illustrating yet another example of the human-computer user interface of the data processing system according to the present invention; and

FIG. 8 is a schematic diagram illustrating still another example of the human-computer user interface of the data processing system according to the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

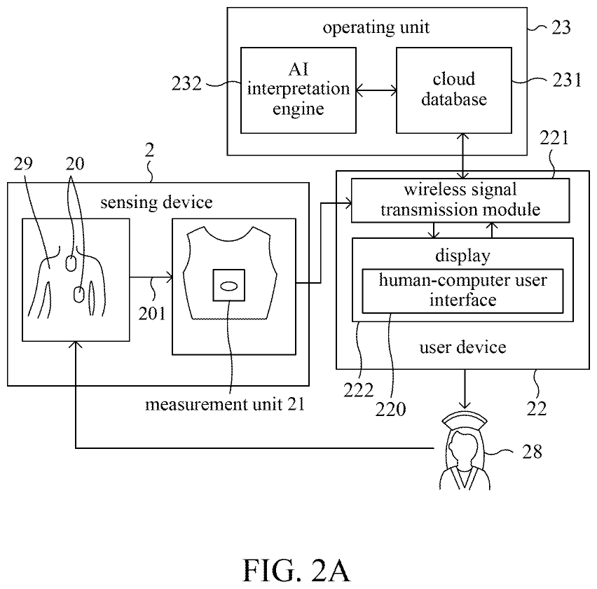

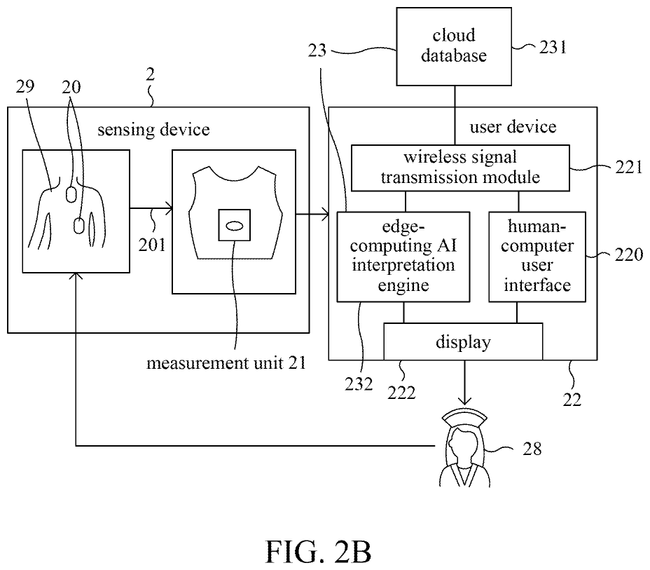

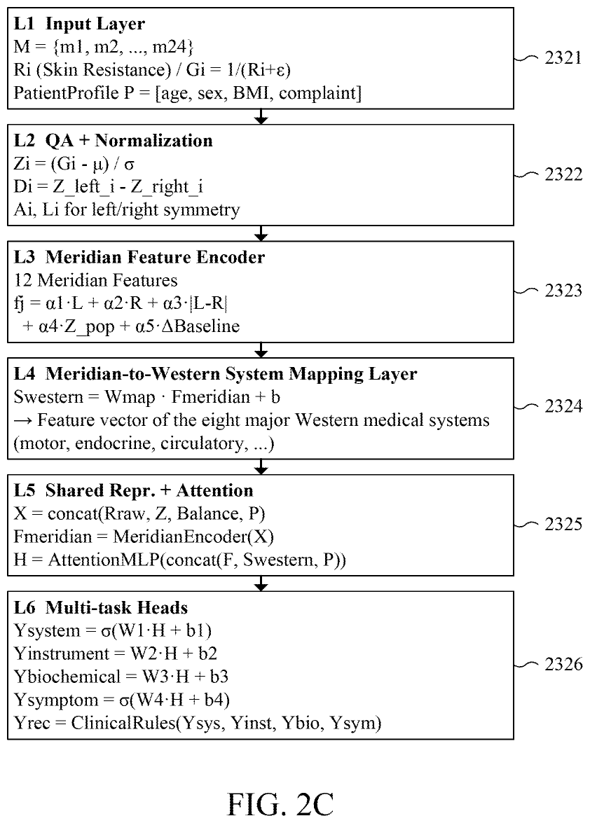

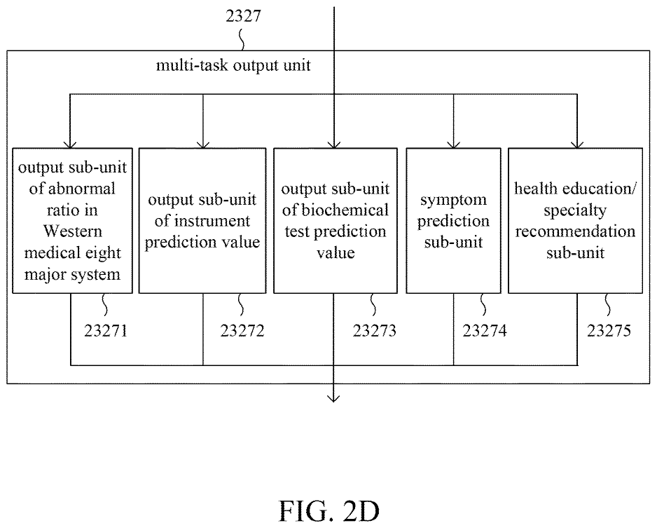

Referring to FIG. 2A, a functional block diagram of a data processing system according to an embodiment of the present invention is schematically illustrated. In this embodiment, the data processing system collects and converts meridian parameters of a patient into biomedical data in an easy and readily available manner. As known, meridian parameters are common and important information for diagnosing a patient in traditional Chinese medicine, while biomedical data are essential to diagnose a patient in Western medicine. In this embodiment, the data processing system includes a sensing device 2 for collecting meridian parameters of a patient 29, an operating unit 23 for processing the collected meridian parameters and a user device 22 interfacing the sensing device 2 and the operating unit 23. The sensing device 2 includes a plurality of sensors 20 and a measurement unit 21. The plurality of sensors 20 may be, for example, sensing patches or probes attached onto the body of the user 22 at effective sensing zones for collecting meridian parameters. The effective sensing zones are predetermined according to theory of traditional Chinese medicine. For example, the plurality of sensing zones 24 include well points related to the twelve meridians (three yin meridians of the hand, three yang meridians of the hand, three yin meridians of the foot and three yang meridians of the foot). A plurality of sensing signals, for example, in a form of electric signals, are generated by the sensors 20 and transmitted to the measurement unit 21 via signal lines 201. The measurement unit 21 then outputs a plurality of meridian signal values, e.g., skin resistance values of well points, corresponding to the sensing signals. In this embodiment, the measurement unit 21 may be implemented with fixed apparatus or a portable device. For example, the measurement unit 21 may be integrated into a wearable device, such as a vast, to be worn by the patient 29 during inspection. Accordingly, the inspection can be conveniently executed, without limiting the inspection site, by taking out the sensors 20, for example, from the vast pocket, attaching the sensors 20 onto the body of the patient 29 and obtaining data from the measurement unit 21. The meridian signal values form a parameter set, e.g., a resistance value array.

Subsequently, the measurement unit 21 transmits the meridian signal values to a wireless signal transmission module 221 of the user device 22 by way of wireless transmission means, such as Bluetooth or WLAN. The user device 22, for example, may be implemented with a smart phone, a tablet or a personal computer, where a human-computer user interface can run. The user device 22 transmits out the parameter set containing the meridian signal values from the wireless signal transmission module 221, and the parameter set is transmitted to a remote cloud database 231 of the operating unit 23, for example, via WLAN or Internet, and stored in the remote cloud database 231. The parameter set can indicate the real-time physiological state of the patient 29. For example, the parameter set can be composed of twenty-four biomedical signal values (e.g., skin resistance values of well points) detected by the measurement unit 21. The related theory and technology can be found in, for example, the literature “Study on the Reproducibility and Consistency Measurement of Skin Resistance at Well Points in the twelve meridians” in the Journal of Traditional Chinese Medicine of North Taiwan 2(1): 1-15, 2020. Hardware devices in connection to the theory and technology have been well developed, and thus need not be redundantly described herein. Unfortunately, it is hard for medical personnel in the field of western medicine to understand the meridian systems defined in the field of Traditional Chinese medicine, including lung meridian, heart meridian, packet meridian, small intestine meridian, triple energizer meridian, large intestine meridian, spleen meridian, liver meridian, kidney meridian, bladder meridian, gallbladder meridian and stomach meridian. Therefore, it is hard to refer to the inspection results obtained by way of Traditional Chinese medicine to assign the patient to a proper medical specialty under Western medicine.

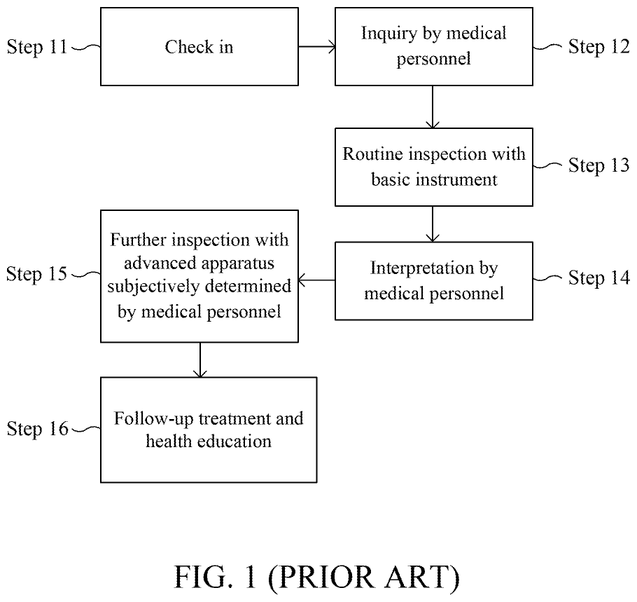

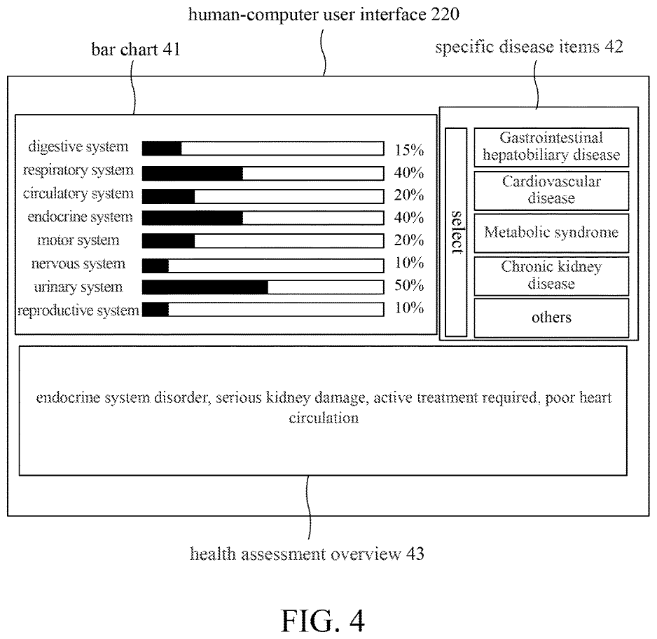

For solving such problems, a data processing system according to the present invention is provided to integrate the inspection results executed under Traditional Chinese medicine and assignment of medical specialty under western medicine. In the embodiment as illustrated in FIG. 1, the operating unit 23 further includes an artificial intelligence (AI) interpretation engine 232. The AI interpretation engine 232 is in communication with the cloud database 231. For assignment of medical specialty, the AI interpretation engine 232 accesses and interprets the parameter set of meridian signal values stored in the cloud database 231, and accordingly, generates a set of biomedical data complying with the definition of systems in Western medicine and, desirably but not necessarily, further generates at least one prediction indicator corresponding to a symptom defined in Western medicine. The systems defined in Western medicine may include motor system, endocrine system, circulatory system, nervous system, digestive system, respiratory system, urinary system and reproductive system. The set of biomedical data generated by the AI interpretation engine 232 may include, for example, a percentage value indicating abnormal probability of a specified system. FIG. 4 exemplifies a set of biomedical data shown on a human-computer user interface 220 on the display 222 of the user device 22. In this example, a bar chart 41 shown on the human-computer user interface 220 includes bars of the following percentage values: motor system 20%, endocrine system 40%, circulatory system 20%, nervous system 10%, digestive system 15%, respiratory system 40%, urinary system 50% and reproductive system 10%. It is realized from the bar chart that abnormal probability is relatively high in endocrine system, respiratory system and/or urinary system. Medical personnel 28 can thus easily refer to the biomedical data indicated by the bar chart 41 to take proper actions. For example, an advanced apparatus, e.g., biochemical analyzer, ultrasonic image analyzer, etc., may be selected for detailed examination.

In the above example, the set of biomedical data shows that the urinary system could be the most probable problem of the illness, followed by the endocrine system and the respiratory system. Therefore, medical personnel 28 may choose the selective item “chronic kidney disease” among the specific disease items 42 shown on the human-computer user interface 220 illustrated in FIG. 4. Accordingly, advanced apparatus would be proposed to perform subsequent advanced examinations directed to the urinary system. In order to avoid misjudgment, follow-up advanced examination can also be carried out on the endocrine system and the respiratory system. From the above description, it is understood that the data processing system in this example facilitates quick judgement of medical staff on subsequent process. Even better, an overview of health assessment for the eight major systems can be further provided based on the set of biomedical data. For example, a text description like “endocrine system disorder, serious kidney damage, active treatment required, poor heart circulation” may be shown in the health assessment overview block 43 as illustrated in FIG. 4.

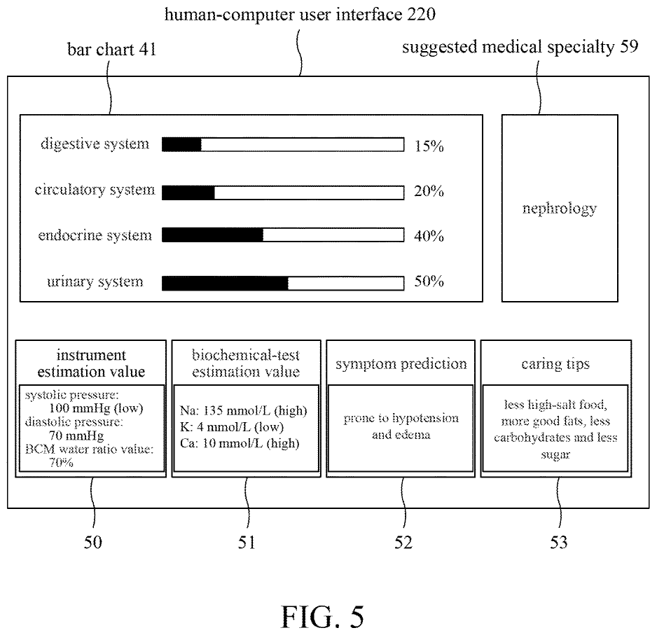

In another embodiment, the interpretation operation carried out by the system according to the present invention may further generate at least one prediction indicator corresponding to specific symptom or symptoms defined under western medicine. For example, as shown in FIG. 5, in which the set of biomedical data is partially shown, the at least one prediction indicator shown the human-computer user interface 220 may include a follow-up treatment proposal 59, e.g., suggested medical specialty, in addition to the bar chart 41 described above (partially shown herein). The follow-up treatment proposal 59 shown in this example may be, for example, “suggested medical specialty: nephrology”. Furthermore, the at least one prediction indicator may include an instrument estimation value, a biochemical-test estimation value, symptom prediction and caring tips, as shown in blocks 50, 51, 52 and 53 of the human-computer user interface 220, respectively. For example, the instrument estimation value shown in block 50 may include the systolic pressure and diastolic pressure of blood pressure and the water ratio value, etc. Generally, in western medicine, the systolic pressure and diastolic pressure of blood pressure are measured by a sphygmomanometer, and the water ratio value is measured by a body composition monitor (BCM). On the contrary, according to the present invention, the systolic pressure and diastolic pressure of blood pressure and the water ratio value are estimated based on the set of biomedical data, which originate from the sensed meridian parameters, without using the sphygmomanometer and the body composition monitor. In the example illustrated in FIG. 5, the estimated systolic pressure and diastolic pressure are 100 mmHg and 70 mmHg, respectively, and the estimated water ratio value is 70%. The estimated value, if abnormal, would be specified for warning. For example, 100 mmHg is relatively low for systolic pressure and thus specifically marked in the human-computer user interface 220 on the display 222. It is then desirable for medical personnel to use the sphygmomanometer to measure the systolic pressure again for reconfirmation.

In another aspect, the biochemical-test estimation value may be, for example, sodium ion (Na⁺) concentration (mmol/L), potassium ion (K⁺) concentration (mmol/L), calcium ion (Ca²⁺) concentration (mmol/L), etc. Any of the estimated values beyond a predetermined normal range will be specifically marked in the human-computer user interface 220 on the display 222. It is then desirable for medical personnel to use a proper instrument to measure the deviated ion concentration(s) for reconfirmation. As for the symptom prediction and the caring tips shown in blocks 52 and 53, they are displayed and specified, if abnormal, for advising possible problems and giving notices for prevention. In the example shown in FIG. 5, the symptom prediction includes “prone to hypotension and edema”, and the caring tips include “less high-salt food, more good fats, less carbohydrates and less sugar”.

In this embodiment, the above-described set of biomedical data and the at least one prediction indicator are obtained by the well-trained AI interpretation engine 232, which accesses, interprets and operates the parameter set of meridian parameters stored in the cloud database 231, and shown on the display 222 of the user device 22 for reference of medical personnel. The obtained set of biomedical data and the at least one prediction indicator may also be stored in the cloud database 231. For training the AI interpretation engine 232, a large amount of training data may be prepared and inputted to the AI interpretation engine 232. The training data are generated by interpreting, judging and tagging real physiological numerical measurements by experienced Chinese medicine practitioners and western medicine practitioners. The generated training data are then stored into the cloud database 231. The training data correlates a plurality of sets of meridian parameters to a plurality of sets of biomedical data, and preferably a plurality of prediction indicators, defined in western medicine. The training algorithm of the AI interpretation engine 232 may include, for example, a compound convolutional neural network and/or an expert system, but not limited thereto. Both the compound convolutional neural network and the expert system can be modified and optimized by reading training data in the cloud database for deep learning. Of course, in addition to deep learning, the training algorithm may also include machine learning and a compound algorithm.

FIG. 2B is a functional block diagram, schematically illustrating a data processing system according to another embodiment of the present invention. Similar to the above embodiment, the data processing system also includes a sensing device 2 for collecting meridian parameters of a patient 29, an operating unit 23 for processing the collected meridian parameters and a user device 22 interfacing the sensing device and the operating unit 23. The sensing device 2 includes a plurality of sensors 20, e.g., sensing patches or probes, which are attached onto the body of the user 22 at effective sensing zones for collecting meridian parameters, and a measurement unit 21, which may be integrated into a wearable device such as a vast to be worn by the patient 29 during inspection. The effective sensing zones are predetermined according to theory of traditional Chinese medicine. For example, the plurality of sensing zones include 24 well points related to the twelve meridians. A plurality of sensing signals, for example, meridian signals in a form of electric signals, are generated by the sensors 20 and transmitted to the measurement unit 21 via signal lines 201. The measurement unit 21 then outputs a plurality of meridian signal values, e.g., skin resistance values of well points, corresponding to the sensing signals. The meridian signal values form a parameter set, e.g., a resistance value array. Subsequently, the measurement unit 21 transmits the parameter set of meridian signal values to the user device 22 by way of wireless transmission means, such as Bluetooth or WLAN. The user device 22, for example, may be implemented with a smart phone, a tablet or a personal computer, where a human-computer user interface 220 can run. The operating unit 23 in this embodiment includes a cloud database 231 and an edge-computing AI interpretation engine 232. The edge-computing AI interpretation engine 232 may be disposed in the user device 22 in this embodiment. The parameter set of meridian signal values transmitted to the user device 22 is further transmitted to the edge-computing AI interpretation engine 232 disposed in the user device 22 to be operated into a set of corresponding biomedical data defined in Western medicine, and preferably, at least one additional prediction indicator. The set of biomedical data and the at least one prediction indicator may be transmitted to the cloud database 231 of the operating unit 23 via the wireless signal transmission module 221 of the user device 22 to be stored in the cloud database 231. The set of biomedical data and the at least one prediction indicator may also be shown on a display 222 of the user device 22. Medical personnel 28 can thus refer to the set of biomedical data and the at least one prediction indicator and take proper actions, e.g., arrange further inspection and/or drawing up treatment strategy, accordingly.

Please refer to FIG. 2C, which is a functional block diagram schematically illustrating a model developed for the artificial intelligence (AI) interpretation engine 232 according to an embodiment of the present invention. The model includes a six-layer architecture, respectively comprising Input Layer 2321, QA+Normalization Layer 2322, Meridian Feature Encoder Layer 2323, Meridian-to-Western System Mapping Layer 2324, Shared Repr.+Attention Layer 2325, and Multi-task Heads Layer 2326. The core function of this model, referred to as Meridian-to-Western System Multi-task Neural Network (MW-MTNN), is to calculate the “meridian data” measured from traditional Chinese medicine through the AI model, and thereby translate and predict “health check indicators” and “lifestyle recommendations” defined in Western medicine.

The Input Layer 2321 is configured to collect a basic patient profile (e.g., age, gender, BMI, and chief complaint) and skin resistance data of the 24 well points. The basic patient profile and the skin resistance data of the 24 well points can be acquired from the cloud database 231 and the sensing device 2. The QA+Normalization Layer 2322 is configured to clean the collected data. Since each individual has different skin characteristics (e.g., dampness levels), the QA+Normalization Layer 2322 normalizes the data to eliminate errors and compares a balance state between the “left side” and the “right side” of the patient body.

Furthermore, the Meridian Feature Encoder Layer 2323 extracts features of the 12 primary meridians to determine which meridian has excessive or deficient energy based on the following formula:

$$f\_j = {α}_{1} \cdot L + {α}_{2} \cdot R + {α}_{3} \vert L - R \vert + {α}_{4} \cdot Z\_pop + {α}_{5} \cdot Δ Baseline$$

wherein “f_j” represents a final extracted comprehensive feature value of the j-th primary meridian (12 in total); “L” and “R” represent measured values of the meridian on the left side and the right side of the body, respectively; “|L−R|” represents an absolute value of a difference between the left and right sides, indicating a degree of left-right imbalance of the meridian; “Z_pop” represents a normalized score (Z-Score) relative to an overall population data group; “ΔBaseline” represents a deviation value of the meridian from the patient's own basic health baseline; and “α₁, α₂, α₃, α₄, α₅” represent weight parameters to be learned by the model, which are used to determine the influence and importance of each feature on the final meridian features.

The Meridian-to-Western System Mapping Layer 2324 converts the meridian data of traditional Chinese medicine into a feature vector of eight major systems defined in Western medicine (e.g., circulatory system, endocrine system, motor system, etc.). The main conversion formula is: S_western=W_map·F_meridian+b, wherein “S_western” represents the feature vector of the eight major Western medical systems (i.e., motor system, endocrine system, circulatory system, nervous system, digestive system, respiratory system, urinary system, and reproductive system); “W_map” represents mapping weights responsible for mapping a meridian feature space to a Western medical system space; “F_meridian” represents a Chinese medicine meridian feature vector combined from the previous layer (Layer 3); and “b” represents a bias, which is an adjustment constant of the neural network.

The Shared Repr.+Attention Layer 2325 is configured to package and fuse all the previously extracted information (Chinese medicine features, Western medicine features, and personal profile) and activate an “attention mechanism”. That is, the AI model is enabled to read the information and automatically focus on the “most critical health issues”. The related formulas are as follows:

$$X = concat ( R\_raw , Z , Balance , P ) ;$$

$$F\_meridian = MeridianEncoder ( X ) ;$$

$$H = AttentionMLP ( concat ( F , S\_western , P ) )$$

wherein “concat” is a concatenation function used to combine multiple vectors of different dimensions into a single comprehensive vector; “X” is a total input feature vector fusing raw resistance, normalized scores, balance features, and the basic patient profile; “R_raw” is a collected raw skin resistance data set; “Z” is a normalized Z-Score vector; “Balance” is a feature indicator representing the balance state of meridians on the left and right sides of the patient's body; “P” is a patient basic profile feature vector (age, gender, BMI, chief complaint); “MeridianEncoder” is a meridian encoder neural network responsible for further extracting the fused feature X into high-level abstract features; “H” is a final output full-function hidden representation feature vector, serving as a prediction basis for all subsequent tasks; “AttentionMLP” is a Multilayer Perceptron with an attention mechanism capable of automatically focusing on the most critical health features; and “F” represents the deep meridian encoded feature vector F\_meridian.

The core mathematical formulas of the Multi-task Heads Layer 2326 include:

$$Y\_system = σ ( W\_ 1 \cdot H + b\_ 1 )$$

$$Y\_instrument = W\_ 2 \cdot H + b\_ 2$$

$$Y\_biochemical = W\_ 3 \cdot H + b\_ 3$$

$$Y\_symptom = σ ( W\_ 4 \cdot H + b\_ 4 )$$

$$Y\_rec = ClinicalRules ( Y\_sys , Y\_inst , Y\_bio , Y\_sym )$$

wherein “Y_system” is a prediction output of abnormal probabilities of the eight major Western medical systems (multi-label classification); “Y_instrument” is a prediction value of continuous clinical indicators (e.g., systolic blood pressure SBP, diastolic blood pressure DBP, blood sugar, body water ratio); “Y_biochemical” is a prediction value of biochemical tests (e.g., sodium Na, potassium K, calcium Ca ion concentrations in blood); “Y_symptom” is a prediction output of clinical symptom occurrence probabilities (e.g., hypotension, edema, headache, muscle weakness); “Y_rec” represents health education recommendations and suggested medical specialties provided by the system (e.g., recommendation to visit a nephrology department, low-salt diet); “σ” is a Sigmoid activation function that compresses values to between 0 and 1 to represent probabilities; “ClinicalRules” is a rule-based expert system that combines multiple prediction results to derive final health education and medical guidelines; “W_1, W_2, W_3, W_4” are independent neural network weight matrices for respective task output heads; “b_1, b_2, b_3, b_4” are independent bias values for respective task output heads; and “Y\_sys, Y\_inst, Y\_bio, Y\_sym” are output abbreviations corresponding to the above prediction heads, serving as input rule indicators for the expert system.

Please refer to FIG. 2D, which is an internal functional block diagram of a multi-task output unit 2327. The multi-task output unit 2327 is configured to receive the output of the Multi-task Heads Layer 2326 and execute multiple tasks respectively using a plurality of sub-units thereof. The multi-task output unit 2327 mainly includes an output sub-unit 23271 of abnormal ratio in a Western medical eight major system, an output sub-unit 23272 of an instrument prediction value, an output sub-unit 23273 of a biochemical test prediction value, a symptom prediction sub-unit 23274, and a health education/specialty recommendation sub-unit 23275, to simultaneously answer five different health-related categories. Specifically, the abnormal ratio output sub-unit 23271 outputs a probability of illness in body systems based on data (e.g., urinary system abnormality 72%, endocrine abnormality 61%, circulatory system abnormality 48%). The instrument prediction value output sub-unit 23272 directly estimates blood pressure (SBP/DBP), blood sugar, and body water ratio. The biochemical test prediction value output sub-unit 23273 estimates ion concentrations such as Na, K, and Ca in the blood. The symptom prediction sub-unit 23274 predicts potential discomforts, such as hypotension, edema, headache, or muscle weakness. Finally, the health education/specialty recommendation sub-unit 23275 provides lifestyle guidelines and advice (e.g., “You should visit the nephrology department,” or “Keep a low-sodium diet”).

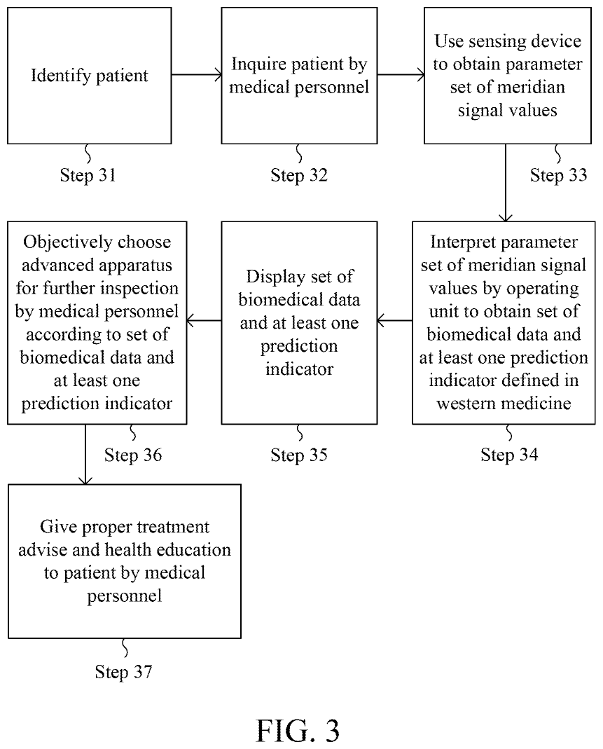

Please refer to FIG. 3, which schematically illustrates a flow of a medical service according to an embodiment of the present invention. After medical personnel identifies the patient in a clinic, a care institution or at home (Step 31), the medical personnel inquires basic state of illness of the patient (Step 32). Then the data processing system according to the present invention is applied onto the patient. First of all, the sensing device is used to obtain the parameter set of meridian signal values (Step 33). The sensing device of the data processing system inspects meridian parameters of the patient and measures a set of meridian parameters values, e.g., skin resistance values of well points, of the patient based on the meridian parameters, and the operating unit interprets the set of meridian parameters values (Step 34). Accordingly, a set of biomedical data and/or at least one prediction indicator defined in western medicine are derived from the set of meridian parameters values and shown to the medical personnel (Step 35). The medical personnel further selects an advanced apparatus to inspect the patient and diagnose a possible disease based on the set of biomedical data and/or at least one prediction indicator, which is relatively objective (Step 36). Subsequently, the medical personnel can give proper treatment advise and health education to the patient (Step 37).

It is understood by those of ordinary skill in the art that pulsating waveforms of human arteries can be converted into pulse conditions in traditional Chinese medicine, and thus corresponding states of twelve meridians can be realized through frequency spectrum analysis. In other words, pulsating waveform signals can be interpreted as meridian signals. Therefore, the sensing zones where the sensors 20 are attached may be one or more arterial areas of the patient 29 instead of the above-mentioned well points of the patient 29. In this embodiment, the pulses at the arterial areas of the patient 29 are sensed by the sensors 20 and corresponding sensing signals, e.g., pulsating waveform signals, are generated and transmitted to the measurement unit 21 via the signal line 201. Likewise, the measurement unit 21 may be integrated into a wearable device, such as a vast, to be worn by the patient 29 during inspection. The measurement unit 21 measures frequency spectrum distribution data of the pulsating waveform signals and stores the parameter set of the frequency spectrum distribution data of the pulsating waveform signals into the cloud database 231 of the operating unit 23. The measurement unit 21 may be implemented with a physiological signal sensor including a pressure sensor, acoustic sensor, electrical sensor or optical sensor. Subsequently, the edge-computing AI interpretation engine 232 accesses and interprets the parameter set stored in the cloud database 231, and accordingly, generates a set of biomedical data complying with the definition of systems in western medicine and, desirably but not necessarily, further generates at least one prediction indicator corresponding to a symptom defined in western medicine. The training algorithm of the edge-computing AI interpretation engine 232 may be similar to that illustrated previously or any other suitable algorithm, and is not to be redundantly described herein.

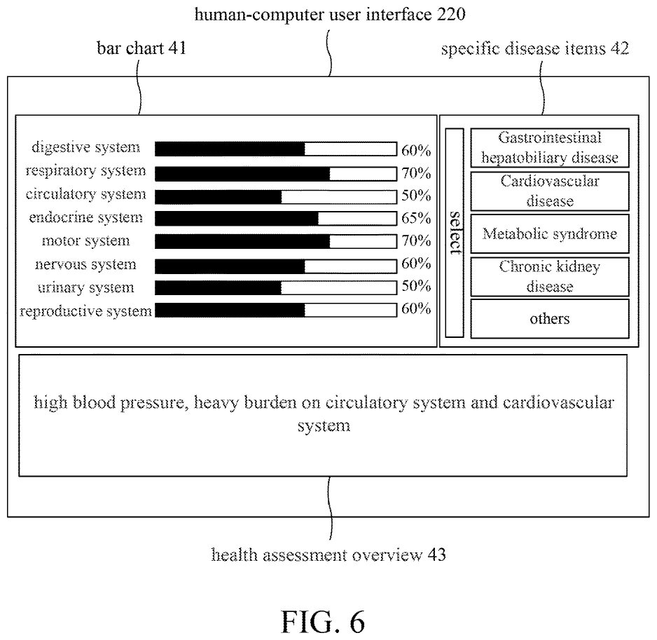

Please refer to FIG. 6, which schematically illustrates a set of biomedical data and at least one prediction indicator shown on the human-computer user interface 220 of the user device 22 after the operating unit 23 receives and interprets the parameter set of the meridian parameters directly measured or derived from the measured frequency spectrum distribution data of the pulsating waveform signals. As exemplified in FIG. 6, the set of biomedical data is indicated by a bar chart 41 shown on the human-computer user interface 220, which includes bars of the following percentage values: motor system 70%, endocrine system 65%, circulatory system 50%, nervous system 60%, digestive system 50%, respiratory system 70%, urinary system 50% and reproductive system 60%. With reference to the set of biomedical data, medical personnel 28 may choose the selective item “cardiovascular disease” among the specific disease items 42 shown on the human-computer user interface 220. Accordingly, advanced apparatus would be proposed to perform subsequent advanced examinations directed to the cardiovascular system. Preferably, an overview of health assessment for the eight major systems can be further provided based on the set of biomedical data. For example, a text description like “high blood pressure, heavy burden on circulatory system and cardiovascular system” may be shown in the health assessment overview block 43 as illustrated in FIG. 6.

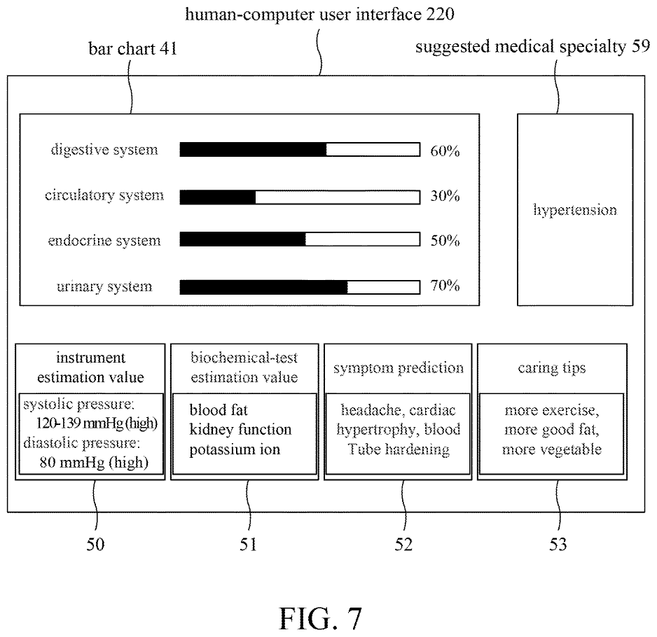

In another embodiment, the interpretation operation carried out by the system according to the present invention may further generate at least one prediction indicator corresponding to specific symptom or symptoms defined under western medicine. For example, as shown in FIG. 7, the at least one prediction indicator shown the human-computer user interface 220 may include a follow-up treatment proposal 59, e.g., suggested medical specialty: hypertension, in addition to the bar chart 41 described above. Furthermore, the at least one prediction indicator may include an instrument estimation value, a biochemical-test estimation value, symptom prediction and caring tips, as shown in blocks 50, 51, 52 and 53 of the human-computer user interface 220, respectively. For example, the instrument estimation value shown in block 50 may include the systolic pressure and diastolic pressure of blood pressure. Generally, in western medicine, the systolic pressure and diastolic pressure of blood pressure are measured by a sphygmomanometer. On the contrary, according to the present invention, the systolic pressure and diastolic pressure of blood pressure are estimated based on the set of biomedical data, which originate from the sensed or derived meridian parameters, without using the sphygmomanometer. In the example illustrated in FIG. 7, the estimated systolic pressure and diastolic pressure are 120-139 mmHg and 80 mmHg, respectively. The estimated value, if abnormal, would be specified for warning. For example, 120-139 mmHg is relatively high for systolic pressure and 80 mmHg is relatively high for diastolic pressure. Thus they are specifically marked in the human-computer user interface 220 on the display 222. It is then desirable for medical personnel to use the sphygmomanometer to measure the systolic pressure and diastolic pressure again for reconfirmation.

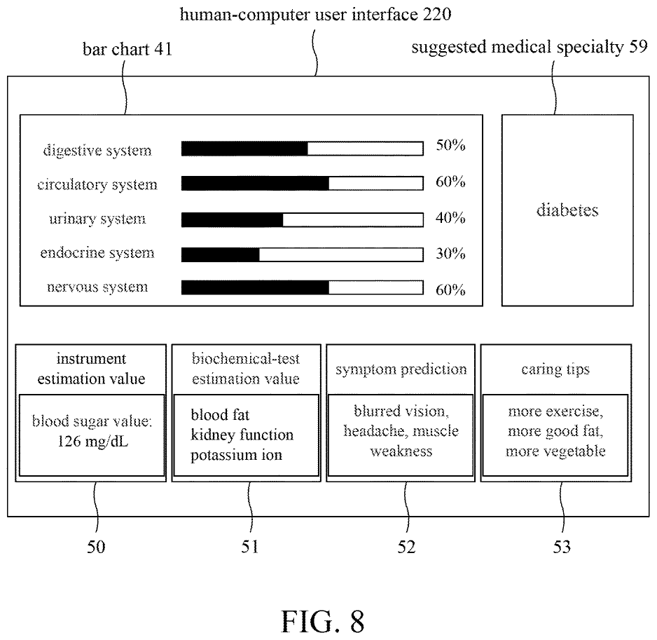

In another example as shown in FIG. 8, the set of biomedical data is shown in the bar chart 41 and a follow-up treatment proposal 59, e.g., suggested medical specialty: diabetes, is also shown as a part of the at least one prediction indicator. The at least one prediction indicator may further include an instrument estimation value, a biochemical-test estimation value, symptom prediction and caring tips, as shown in blocks 50, 51, 52 and 53 of the human-computer user interface 220, respectively. For example, the instrument estimation value shown in block 50 may include a blood sugar value. In this embodiment, the blood sugar value is estimated according to the set of biomedical data without using a blood-glucose meter. In the example illustrated in FIG. 8, the estimated blood sugar value is 126 mg/dL. The estimated value, if abnormal, would be specified for warning. For example, 126 mg/dL is relatively high for the blood sugar value. Thus it is specifically marked in the human-computer user interface 220 on the display 222, so that medical personnel may reconfirm the value, for example, by using a blood-glucose meter.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A data processing system, comprising:
a plurality of sensors to be attached onto a body of a subject at a plurality of sensing zones to generate a plurality of sensing signals;
a measurement unit in communication with the plurality of sensors for measuring the plurality of sensing signals and generating a parameter set containing a plurality of meridian signal values;
an operating unit in communication with the measurement unit, receiving the parameter set and performing an operation of the parameter set to generate a set of biomedical data, wherein the operation includes mapping a meridian feature space to a Western medical system space with a mapping weight matrix; and
a display in communication with the operating unit for showing the set of biomedical data.

2. The data processing system according to claim 1, wherein the plurality of sensing zones comprise a plurality of well points related to meridians defined in traditional Chinese medicine, and the plurality of sensing signals comprise a plurality of meridian signals.

3. The data processing system according to claim 1, wherein the plurality of sensing zones comprise one or more arterial areas, and the plurality of sensing signals comprise a plurality of pulsating waveform signals.

4. The data processing system according to claim 1, wherein the plurality of sensors are patches or probes.

5. The data processing system according to claim 1, wherein the operating unit further performs an operation of the parameter set to generate at least one prediction indicator.

6. The data processing system according to claim 1, wherein the at least one prediction indicator comprises follow-up treatment proposal, health assessment overview, instrument estimation value, biochemical-test estimation value, symptom prediction and/or caring tips.

7. The data processing system according to claim 1, wherein the set of biomedical data comprises an abnormal probability of at least one specified system defined in western medicine.

8. The data processing system according to claim 7, wherein the at least one specified system is selected from one or more of motor system, endocrine system, circulatory system, nervous system, digestive system, respiratory system, urinary system and reproductive system.

9. The data processing system according to claim 1, wherein the measurement unit is integrated into a wearable device.

10. The data processing system according to claim 1, wherein the display is disposed in a user device.

11. The data processing system according to claim 10, wherein the user device is a smart phone, a tablet or a personal computer.

12. The data processing system according to claim 10, wherein the operating unit comprises a cloud database and an artificial intelligence interpretation engine.

13. The data processing system according to claim 12, wherein the artificial intelligence interpretation engine is in communication with the user device via a wireless signal transmission module.

14. The data processing system according to claim 12, wherein the artificial intelligence interpretation engine is an edge-computing artificial intelligence interpretation engine disposed in the user device.

15. The data processing system according to claim 12, wherein the artificial intelligence interpretation engine is a compound convolutional neural network and/or an expert system modified and optimized by reading training data in the cloud database for deep learning.
